(19) United States
(12) Patent Application Publication
(10) Pub. No.: US 2025/0352756 A1
(43) Pub. Date: Nov. 20, 2025

(54) SLEEP CHAMBER FOR SIMULATING RIDING IN A MOTOR VEHICLE

(71) Applicant: Reginald Bovil, San Antonio, TX (US)

(72) Inventor: Reginald Bovil, San Antonio, TX (US)

(21) Appl. No.: 18/665,745

(22) Filed: May 16, 2024

(51) Int. Cl. A61M 21/00; A61M 21/02

(52) U.S. Cl. CPC A61M 21/0094; A61M 21/02; A61M 2021/0022; A61M 2021/0027; A61M 2205/3592; A61M 2205/8206; A61M 2205/8237

(57) ABSTRACT

A sleep pod apparatus for simulating riding in an operating motor vehicle to soothe an occupant of the sleep chamber to sleep includes a housing which defines an interior space therein and defines an opening to the interior space. A processor and a vibration motor are mounted in the housing. The vibration motor is operatively coupled to the processor and is configured to vibrate the cushion when activated by the processor. The vibration motor is configured to simulate a vibration of a motor vehicle on a passenger thereof.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to sleep chambers and more particularly pertains to a new sleep chamber for simulating riding in an operating motor vehicle to soothe an occupant of the sleep chamber to sleep.

### (2) Description of Related Art Including Information Disclosed Under 37 CFR 1.97 and 1.98

Various sleep chambers are disclosed by the prior art, including those which move and play audio to sooth an occupant to sleep. However, the prior art fails to describe such an apparatus which is configured to simulate riding in a moving motor vehicle.

### BRIEF SUMMARY OF THE INVENTION

An embodiment of the disclosure meets the needs presented above by generally comprising a housing which defines an interior space therein and defines an opening to the interior space. A processor and a vibration motor are mounted in the housing. The vibration motor is operatively coupled to the processor and is configured to vibrate the cushion when activated by the processor. The vibration motor is configured to simulate a vibration of a motor vehicle on a passenger thereof.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a sleep pod apparatus according to an embodiment of the disclosure.

FIG. 2 is a side view of an embodiment of the disclosure.

FIG. 3 is an end view of an embodiment of the disclosure.

FIG. 4 is a top in-use view of an embodiment of the disclosure.

FIG. 5 is a block diagram of an embodiment of the disclosure.

### DETAILED DESCRIPTION OF THE INVENTION

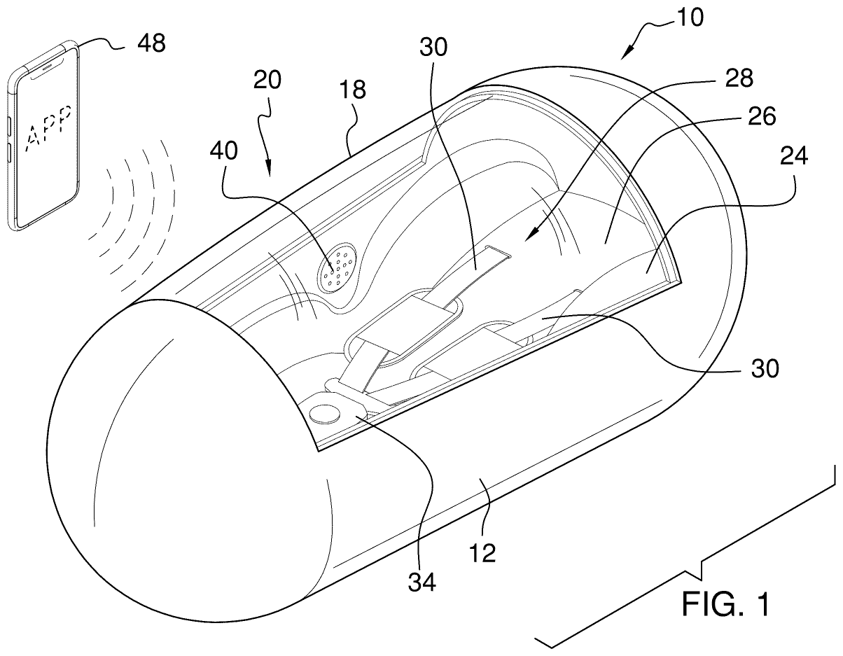

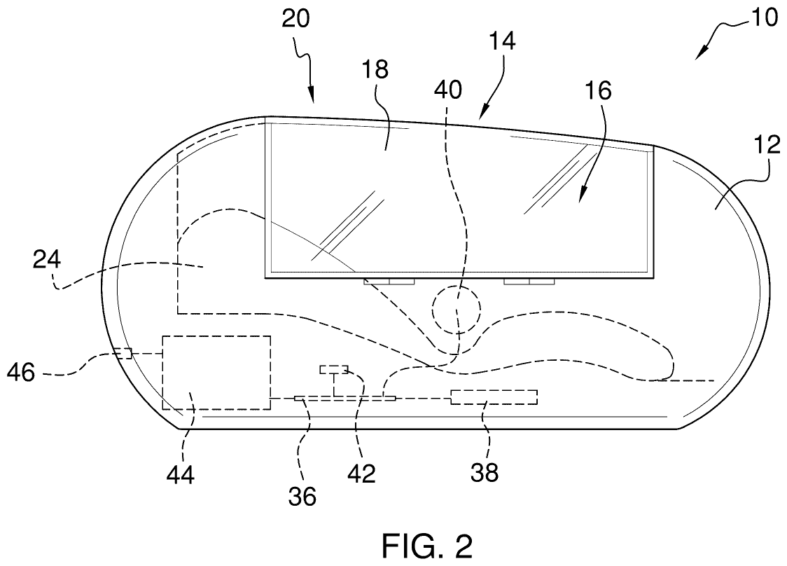

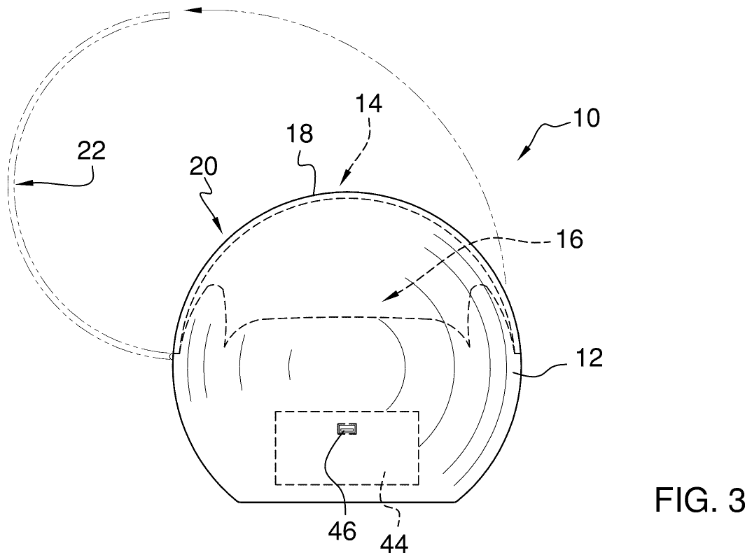

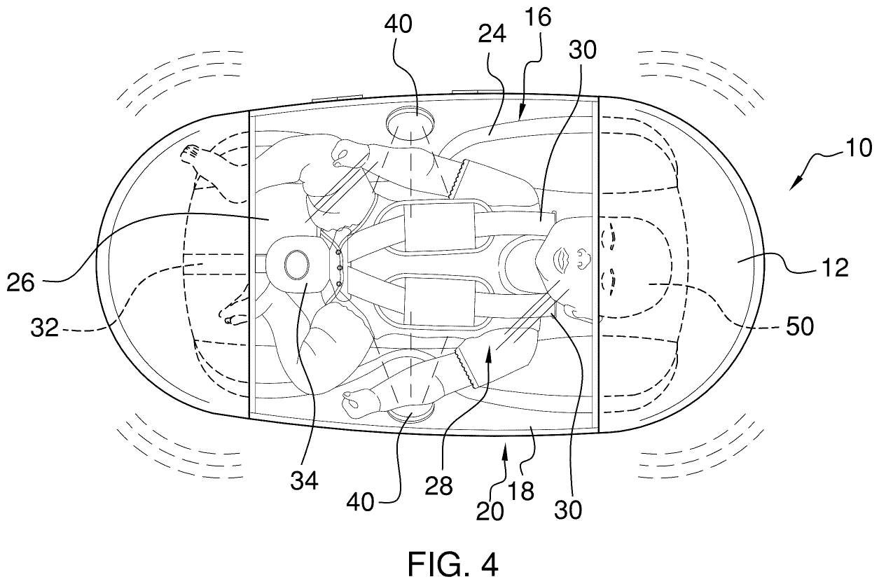

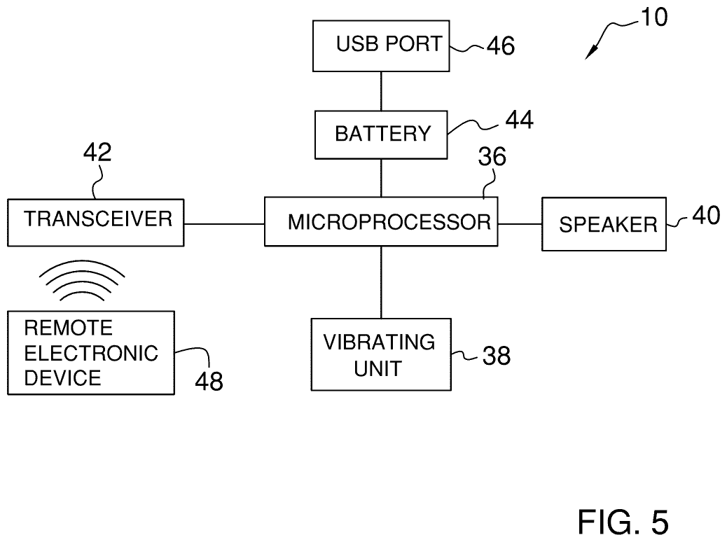

With reference now to the drawings, and in particular to FIGS. 1 through 5 thereof, a new sleep chamber embodying the principles and concepts of an embodiment of the disclosure and generally designated by the reference numeral 10 will be described.

As best illustrated in FIGS. 1 through 5, the sleep pod apparatus 10 generally comprises a housing 12 which defines an interior space 14 therein sized to contain a child 50. The housing 12 defines an opening 16 to the interior space 14, and a cover 18 is pivotably coupled to the housing 12 adjacent to the opening 16. The cover 18 is movable between a closed position 20 and an open position 22 with respect to the opening 16. The cover 18 comprises a transparent material such as a transparent plastic, a glass, or the like, such that the interior space 14 is viewable from outside the housing 12 through the cover 18. A cushion 24 is mounted in the housing 12 and has an upper surface 26 such that the cushion 24 is configured to support the child 50. A harness 28 comprising a pair of shoulder straps 30, a crotch strap 32, and a buckle 34 which selectively joins the shoulder straps 30 to the crotch strap 32 is attached to the cushion 24 over the upper surface 26. Vents may be included in the housing 12 to provide ventilation between the interior space 14 and an exterior of the housing 12 when the cover 18 is in the closed position 20. Fans or other air moving means may be used to further facilitate ventilation between the interior space 14 and the exterior of the housing 12.

A processor 36, a vibration motor 38, speakers 40, and a transceiver 42 are mounted in the housing 12. The processor 36 is configured to operate the vibration motor 38, the speakers 40, and the transceiver 42. The vibration motor 38 is mounted adjacent to the cushion 24 and is configured to vibrate the cushion 24 when activated by the processor 36. When activated, the vibration motor 38 vibrates in a manner which simulates vibrations a passenger of a motor vehicle experiences when the motor vehicle vibrates due to running the engine of the motor vehicle and driving the motor vehicle over a road surface. The speakers 40 are mounted in the interior space 14 of the housing 12, and the processor 36 is configured to selectively play road noise audio via the speakers 40. Road noise audio refers to audio which is similar to that heard by a passenger of a motor vehicle as the motor vehicle drives over a road surface. The road noise audio may be recorded audio, synthesized audio, or a combination thereof. The speakers 40 may also be activated by the processor 36 to play music or other audio different from the road noise audio.

The transceiver 42 is configured to facilitate wireless communication between the processor 36 and a remote electronic device 48. The wireless connection between the transceiver 42 and the remote electronic device 48 may follow Bluetooth protocols, WiFi protocols, or any other suitable wireless communication protocol. The remote electronic device 48 may have an application stored thereon such that a user may input commands to the remote electronic device 48 to operate the processor 36. The processor 36 may also send operational data to the remote electronic device 48, such as an indication of whether the vibration motor 38 and speakers 40 are activated and the audio that is being played via the speakers 40 if any. Audio for playing via the speakers 40 may be stored on a memory of the processor 36, on the remote electronic device 48, or a remote data storage accessible by the processor 36.

A display may be mounted to or integrated with the housing 12 or the cover 18. The display may display the operational data. An input interface may also be mounted to or integrated with the housing 12 or the cover 18. The display and the input interface may also be integrated with each other in the form of a touchscreen or the like. Additional sensors and other electronic components may be included, such as a heart rate sensor which is configured to detect a heart rate of the child 50 when the child 50 is in the interior space 14 of the housing 12. The heart rate sensor may comprise a microphone or other suitable sensing means. The heart rate of the child 50 may be displayed on the display and on the remote electronic device 48.

A power supply 44 is electrically coupled to the processor 36 and is mounted in the housing 12. The power supply 44 comprises a battery but may comprise a capacitor or other suitable energy storage. A charging port 46 is mounted to the housing 12 and is electrically coupled to the power supply 44. The charging port 46 is configured to charge the power supply 44 when electrically coupled to an external power source. The charging port 46 may be a Universal Serial Bus (USB) style port or any other suitable electrical port. In some embodiments, an electrical cord may directly couple the processor 36 to the external power source.

In use, the child 50 is placed in the interior space 14 of the housing 12 on the cushion 24. The child 50 may be strapped into place via the harness 28. The processor 36 is operated via the remote electronic device 48 or the input interface to activate the vibration motor 38 and the speakers 40 as desired to simulate to the child 50 the experience of riding in a motor vehicle. The simulation takes place with a goal to soothe the child 50, including possibly guiding the child 50 toward sleep. The child 50 may be visually monitored through the cover 18 when the cover 18 is in the closed position 20. The heart rate of the child 50 may also be monitored via the heart rate sensor. Additional sensors may be used to monitor other parameters related to the health and physical state of the child 50.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A sleep pod apparatus comprising:
a housing defining an interior space therein, the housing defining an opening to the interior space;
a processor mounted to the housing; and
a vibration motor operatively coupled to the processor and mounted to the housing, the vibration motor being configured to vibrate the cushion when activated by the processor, the vibration motor being configured to simulate a vibration of a motor vehicle on a passenger thereof.

2. The apparatus of claim 1, further comprising a speaker operatively coupled to the processor and mounted in the interior space of the housing.

3. The apparatus of claim 2, wherein the processor is configured to selectively play road noise audio via the speaker.

4. The apparatus of claim 1, further comprising a transceiver operatively coupled to the processor, the processor being configured to wirelessly communicate with a remote electronic device via the transceiver.

5. The apparatus of claim 1, further comprising a power supply electrically coupled to the processor and mounted in the housing.

6. The apparatus of claim 5, wherein the power supply comprises a battery.

7. The apparatus of claim 5, further comprising a charging port mounted to the housing and electrically coupled to the power supply.

8. The apparatus of claim 1, further comprising a cushion mounted in the housing, the cushion having an upper surface such that the cushion is configured to support an occupant.

9. The apparatus of claim 1, further comprising a cover movable between a closed position and an open position with respect to the opening of the housing.

10. The apparatus of claim 9, wherein the cover is pivotably coupled to the housing.

11. The apparatus of claim 9, wherein the cover comprises a transparent material.

12. A sleep pod apparatus comprising:
a housing defining an interior space therein, the housing defining an opening to the interior space;
a cushion mounted in the housing, the cushion having an upper surface such that the cushion is configured to support an occupant;
a cover movable between a closed position and an open position with respect to the opening of the housing, the cover being pivotably coupled to the housing, the cover comprising a transparent material;
a processor mounted to the housing;
a vibration motor operatively coupled to the processor and mounted to the housing, the vibration motor being configured to vibrate the cushion when activated by the processor, the vibration motor being configured to simulate a vibration of a motor vehicle on a passenger thereof;
a speaker operatively coupled to the processor and mounted in the interior space of the housing, the processor being configured to selectively play road noise audio via the speaker;
a transceiver operatively coupled to the processor, the processor being configured to wirelessly communicate with a remote electronic device via the transceiver;
a power supply electrically coupled to the processor and mounted in the housing, the power supply comprising a battery; and
a charging port mounted to the housing and electrically coupled to the power supply.
